(19) United States
(12) Patent Application Publication
(10) Pub. No.: US 2025/0069692 A1
(43) Pub. Date: Feb. 27, 2025

(54) FECAL SAMPLE, BREATH SAMPLE COLLECTION AND ANALYSIS FOR TREATING INFLAMMATORY BOWEL DISEASE

(71) Applicant: Cylinder Health, Inc., Chicago, IL (US)

(72) Inventors: Kimon ANGELIDES, Houston, TX (US); Eric P. MIRABEL, Houston, TX (US)

(73) Assignee: Cylinder Health, Inc., Chicago, IL (US)

(21) Appl. No.: 18/898,171

(22) Filed: Sep. 26, 2024

Related U.S. Application Data
(63) Continuation of application No. 17/034,041, filed on Sep. 28, 2020.
Application No. 17/034,041 is a continuation of application No. 16/735,734, filed on Jan. 7, 2020, now Pat. No. 10,825,550.
Application No. 17/034,041 is a continuation-in-part of application No. 16/735,427, filed on Jan. 6, 2020, now Pat. No. 11,170,888.
(60) Provisional application No. 62/811,429, filed on Feb. 27, 2019.

(51) Int. Cl. G16B 25/10; G16H 20/60

(52) U.S. Cl. CPC G16B 25/10; G16H 20/60

(57) ABSTRACT

The invention is related to a system for treating irritable bowel syndrome, Crohn's disease, ulcerative colitis (collectively, inflammatory bowel disease, or “IBD”) by determining a number of indicators, including genetic markers, gene expression levels, levels of certain compounds in the gut or feces, hydrogen and/or methane levels, and concentrations of particular bacteria in the gut or feces, and correlating one or more such indicators with symptoms in test subjects with IBD; and correlating diet, drugs, supplements or other therapy, with alleviation of IBD symptoms. The correlations established in the test subjects are confirmed or refuted for individuals suffering IBD, and the treatments established as reducing symptoms are supported through messaging and compliance is verified by monitoring the indicators.

CROSS-REFERENCE

This application is a continuation of U.S. patent application Ser. No. 17/034,041, filed Sep. 28, 2020; which is a continuation of U.S. patent application Ser. No. 16/735,734, filed Jan. 7, 2020, now U.S. Pat. No. 10,825,550, issued Nov. 3, 2020; which claims priority from U.S. Patent Application Ser. No. 62/811,429, filed Feb. 27, 2019; and U.S. patent application Ser. No. 17/034,041 is also a continuation-in-part of U.S. patent application Ser. No. 16/735,427, filed Jan. 6, 2020, now U.S. Pat. No. 11,170,888, issued Nov. 9, 2021; all of which are incorporated herein by reference in their entirety.

### BACKGROUND

As much as 74% of the American population experiences various digestive problems and/or conditions. Chronic digestive diseases (such as celiac disease, Crohn's disease, irritable bowel syndrome, or ulcerative colitis) affect at least 70 million people. Furthermore, up to 37% of patients with chronic digestive disease are admitted into an emergency room every year and 70% of such patients need some type of surgical intervention.

Anyone facing a chronic disease or consistent discomfort must typically rely on health care professionals and other sources, like the internet, including blogs, to assess various treatment alternatives. The public information is typically derived from medical records, publications, and/or from structured information derived from questionnaires and structured interviews by health care professionals. Such data and information from questionnaires and structured interviews is limited and often does not capture the more nuanced responses of the participating individuals.

Although people share the same disease or the same chronic symptomology, their ability to navigate through the symptoms often will include personal adjustments to their daily routines. Many people facing chronic diseases and symptoms will try alternative approaches that they are unwilling to share with their physicians so that they do not appear in their medical records or in any of the structured data used to map treatment plans and their success.

One such group of chronic illness, affecting as much as 74% of the American population, are chronic digestive diseases such as celiac disease, Crohn's disease, irritable bowel syndrome, or ulcerative colitis (collectively, “inflammatory bowel disease” or “IBD”) affect at least 70 million people. Such alternative treatments for IBD abound, including herbal remedies, supplements, acupuncture or acupressure, relaxation techniques, or exercise regimes such as yoga or stretching. Patients may also unknowingly and unwittingly expose themselves to effective treatments, and then repeat certain activities or consume certain products, to continue to reap the benefits; while not understanding the portion of the activity or the active ingredients in products which is associated with symptom relief. The effectiveness of all such alternative therapies is unknown, and the IBD patient must try a wide array of such therapies and then try to decide which worked and which did not. Making such determinations is difficult, due to the lack of objectivity in personal observation, and the lack of control (i.e., failure to exclude other possibly relevant factors) in such self-directed analysis.

Evaluating effective alternative IBD treatments is more difficult than making wise dietary choices can be confusing, particularly if certain foods, stress, and/or exercise exacerbate the problem. For individuals with chronic digestive complications or discomfort making everyday food choices, can be confusing, unpredictable, and often embarrassing (in the case of inadvertent public gas or stool discharge). Medical regimens to address these diseases and assist in relieving symptoms are complicated and involve complicated medication regimens that are difficult and expensive to manage. Changes in diet can ameliorate or sometimes even eliminate the symptoms of these conditions. Methane and hydrogen production can be used as markers to determine the effect of dietary changes on symptoms. Symptom relief has been achieved with a low-FODMAP diet (low in fermentable sugars) in a large majority of functional gastrointestinal disorders patients with fructose or lactose intolerance. Wilder-Smith et al., “Predictors of response to a low-FODMAP diet in patients with functional gastrointestinal disorders and lactose or fructose intolerance.” Aliment Pharmacol Ther 2017 April: 45 (8): 1094-1106.

Nevertheless, it is difficult for a patient to determine which foods may not be low-FODMAP. It is even more difficult to be sure a treatment is in fact effective.

It would be markedly advantageous if the effectiveness of treatments could be determined based on a set of indicators which could be monitored to indicate the relative state of the subject's health.

### SUMMARY

As a first step in evaluating an alternative therapy, one would find correlations, in patient feces, between certain genetic markers indicative of mutant subspecies of the bacteria in the gut, certain levels of gene expression, certain levels of volatile organic compounds (VOCs) and certain levels of methane and hydrogen in patient breath (or feces), certain levels of gut bacteria making up the microbiome; and negative symptomology (“Events”), as determined in a group of test subjects. After establishing those correlation, one can use them in finding therapies and foods which prevent, reduce incidence of or otherwise ameliorate Events and symptoms, and finding in test subjects correlations between consuming such foods and/or practicing such therapies and levels (i) of volatile organic compounds (VOCs) in patient feces, and levels (ii) of methane and hydrogen in patient breath (or feces), and optionally, (iii) microbiome composition (including as determined from fecal genetic markers indicating mutants or wild type, analyzed by either DNA or 16 S RNA analysis), or (iv) gene expression; so that levels (i), (ii), (iii) and/or (iv) can serve as indicators of a therapy and/or diet which reduces Events. Where a subject has levels of (i), (ii), (iii) and/or (iv) indicating that the alternative therapy is not efficacious, and reports a reduction in frequency of Events, this can indicate the subject is being benefited from some other means than the therapy. Such subjects can be queried to try to find the source of their symptom relief, and it can be subject to the same analysis against levels of (i), (ii), (iii) and/or (iv) as above to verify or refute its effectiveness. As a further step, the molecular-level microbiome analysis can identify changes in gut bacteria over time, and correlate those changes with changes in levels (i) or (ii) above, and with symptom relief. Thus, mutant bacteria associated with symptoms can be identified from fecal samples.

Embodiments of the invention further include systems and methods for capturing crowd wisdom to be tested for individualized treatment plans. These systems and methods include data mining crowd sourced health related information and unstructured medical narratives and storytelling from patients to identify treatment plans and general techniques that individuals with chronic diseases/symptoms, including IBD, use to improve their general health and wellbeing. The crowd wisdom is captured by an information management system that obtains input from affected individuals about their preferred treatments, and from published information sources.

A related embodiment of the invention captures crowd wisdom by monitoring of subjects' parameters; including, both objective parameters and the self-reported parameters, in those subjects who report periodic or episodic symptom relief but are unaware of the source of relief. During periods of symptom relief, as verified by one or more of the parameters, the subjects' food and drug consumption, activities, travel, home environment, and other variables can be monitored to determine if the relief can be tied to any such variables.

The system of the invention provides a system to find potential treatments from public literature and from subjects themselves; determine indicators of IBD; verify the correlation of those indicators to a disease or amelioration state in an individual; place the individual on an unapproved treatment regime; and then verify or refute the treatment regime's effectiveness both by its effect on symptoms as reported by the individual, and by whether the objective indicators follow the expected pattern and correlate with the reported symptoms. Messages are preferably sent to reinforce compliance with the treatment regime, if the indicators do not follow the expected pattern and correlate with the reported symptoms.

The systems of the invention can be used in a similar manner to verify or refute treatments for other chronic conditions, besides IBD; provided a set of indicators for the status of the condition can be determined. These systems allow filtering out of ineffective treatments or disproving of correlations which don't in fact exist; such as “vaccines cause autism.” Such ineffective treatments or nonexistent correlations may be widely touted and disseminated on the web or by social media; and therefore, widely accepted Finding effective treatments and extant correlations to disease states is a significant public health benefit.

The therapy further includes finding a diet which ameliorates Events in test subjects with genetic markers or levels of gene expression correlated with IBD; and then applying that diet to participants with the same markers or gene expression levels.

After finding such an optimal diet for the test subjects, the diet is tried in an individual, and changed as necessary to reduce Events and/or change levels (i), (ii), (iii) and/or (iv) to more desirable levels—with the goal being to prevent, reduce incidence of or ameliorate Events in the individual.

Test subjects and participants send in fecal samples preferably collected with a collection system as outlined in FIGS. 1-4C; or otherwise, for testing for levels (i), (ii), (iii) and/or (iv). For preferably determining levels of (ii), test subjects and participants periodically measure methane and hydrogen in the breath, using a wireless device which sends the results to a server. See e.g., U.S. Publ'n No. 20180271404 (disclosing a methane and hydrogen sensor for breath, to integrate with a smartphone or other device). Test subjects and participants report their symptoms and food intake, preferably using a wireless device which sends the results to the server. Alternatively, the off-gassed methane and hydrogen in a fecal sample can be measured.

The server includes software agents which analyze the input from test subjects and participants against a profile they initially provided, and sends messages regarding foods to avoid or preferentially consume, based on all the available information. The software agents preferably monitor the effect of particular messages on particular test subjects or participants, in terms of moving their food consumption to one which is more preferred, and sends the more effective messages going forward to those. The server preferentially also determines if participants are accurately reporting their food consumption, by monitoring the reported food consumption against levels (i), (ii), (iii) and/or (iv), and determining if there is the predicted correlation. If the predicted correlation is lacking, the participant is assumed to not be accurately reporting food consumption; and can be sent a message to report more accurately.

Fecal samples must be initially monitored for markers and gene expression levels, and baseline VOCs. Initial breath samples are monitored for methane and hydrogen levels. Fecal samples are then periodically monitored for changes to genetic markers, gene expression levels, baseline VOCs, and breath samples are periodically monitored for methane and hydrogen levels. A preferred sample collection system includes a disposable feces catcher to place below the toilet seat, and a sealed, pre-addressed sample collection tube, which has a self-sealing cap through which a probe with sample adhered at one end, is pushed into the interior of the tube.

An alternative fecal sample container has a multi-chamber design and can be used to determine the quantity of hydrogen and/or methane from a fecal sample, instead of or in addition to the measurement of hydrogen and/or methane from the subject's breath. In this alternative, a relatively standard amount of fecal material is placed in the container and the off gas is collected—preferably in a chamber of the same sample container. The off gas can also be analyzed to determine the levels of VOCs in the fecal sample.

In some embodiments, as a first step in evaluating alternative therapies, one would review the patients in a database and determine which treatments generated symptom relief, or which generated positive changes in the microbiome. In humans, the gut contains the largest numbers of bacteria and the greatest number of species of bacteria of any area of the human body. An individual's general health and wellbeing is dependent on the proper balance of the bacterial populations in the gut. Imbalances in the intestinal bacterial flora are associated with a number of digestive and immunological disorders. The presence or overabundance of certain types of bacteria have been reported to contribute to obesity, inflammatory bowel diseases, irritable bowel syndrome and other inflammatory or autoimmune conditions. The microbiome composition can thus be an objective indicator of effectiveness of alternative therapies.

Symptom relief can be measured by querying the patient, or by monitoring a number of parameters, including factors like clinical and psychosocial conditions: adherence to recommended medication and nutrition for IBD; biometrics at the point of care; usage & engagement in activities; absenteeism, presenteeism, medical and pharmacy claims history; and medical records. In addition to the microbiome composition, other objective indicators can indicate effectiveness of alternative therapies, including gut methane and hydrogen production, as well as other volatile compounds.

Regarding methane and hydrogen production, animal model experiments have shown that methane, a gaseous by-product of intestinal bacteria, slows small intestinal transit and appears to do so by augmenting small bowel contractile activity. Pimentel M, et al., “Methane, a gas produced by enteric bacteria, slows intestinal transit and augments small intestinal contractile activity.” Am J Physiol Gastrointest Liver Physiol 2006, 290: G1089-95. In the lactulose breath test (where the patient is challenged with lactulose and then methane production is measured), methane in the breath of IBS patients has been associated with severity of constipation. Chatterjee S, “The degree of breath methane production in IBS correlates with the severity of constipation.” Am J Gastroenterol 2007; 102:837-41. Elevated hydrogen production, as measured in the breath, is also widely believed to be associated with symptoms in inflammatory bowel disease.

The levels of certain volatile organic metabolites in the feces of patients with diarrhea-predominant IBS (IBS-D), active Crohn's disease (CD), ulcerative colitis (UC) (collectively, inflammatory bowel disease, or “IBD”) and healthy controls are indicators of IBD. Ahmed, I. et al. “An Investigation of Fecal Volatile Organic Metabolites in Irritable Bowel Syndrome,” PLOS One. 2013; 8 (3): e58204. These researchers arrived at a list of 28 such volatile metabolites (“VOCs”) associated with IBS and not healthy controls, and a list of 11 such volatile metabolites associated with healthy controls and not with IBS.

TABLE A

| No. | Compounds |
|---|---|
| 1 | Butanoic acid, ethyl ester |
| 2 | Propanoic acid, methyl ester |
| 3 | 1-Methyl-2-(1-methylethyl)-benzene |
| 4 | Butanoic acid, butyl ester |
| 5 | Butanoic acid, propyl ester |
| 6 | Hexanoic acid, methyl ester |
| 7 | Propanoic acid, propyl ester |
| 8 | Acetic acid, butyl ester |
| 9 | Butanoic acid, 3-methyl-, butyl ester |
| 10 | Propanoic acid, butyl ester |
| 11 | Cyclohexanecarboxylic acid, ethyl ester |
| 12 | Butanoic acid, 2-methyl-, propyl ester |
| 13 | Ethanoic acid, ethyl ester |
| 14 | Pentanoic acid, 4-methyl |
| 15 | Acetic acid, pentyl ester |
| 16 | Pentanoic acid, butyl ester |
| 17 | Butanoic acid, 3-methyl-, propyl ester |
| 18 | Cyclohexanecarboxylic acid, propyl ester |
| 19 | 6-Methyl-5-hepten-2-one |
| 20 | Propanoic acid, 3-methyl-butyl ester |
| 21 | Ethanoic acid, 3-methyl-1-butyl ester |
| 22 | Cyclohexanecarboxylic acid, butyl ester |
| 23 | Benzoic acid, 2-hydroxy-, methyl ester |
| 24 | Pentanoic acid, 4-methyl-, pentyl ester |
| 25 | Butanoic acid, 3-methyl-, methyl ester |
| 26 | Thiopivalic acid |
| 27 | 5-Methyl -2-(1-methylethyl)-cyclohexanone |
| 28 | 4-Methyl-1-Indole |

TABLE B

| | |
|---|---|
| 1 | 2-Heptanone |
| 2 | 2-Methylpropanal |
| 3 | 3-Methylbutanoic acid |
| 4 | Undecane |
| 5 | 3-Methylbutanal |
| 6 | 2-Methylpropanoic acid |
| 7 | 2-Methyl-1-propanol |
| 8 | 1R-a-Pinene |
| 9 | 2-Penhifizran |
| 10 | Methoxy-phenyl-oxime |
| 11 | 2-Methylfuran |

These compounds could be detected in a fecal sample to indicate the presence of IBD, or the likelihood that it is in remission or symptoms have alleviated (where the compounds in Table B predominate). More importantly, they could be used to determine the effectiveness of alternative therapies and an appropriate diet for amelioration of IBD, by determining which therapies and foods cause increases or decreases in these volatile metabolites, first in a group of test subjects using Al/software agents to find the optimal foods, then in each individual who would be a participant, who could be monitored for these metabolites while using the alternative therapy and controlling for other factors like diet, which could affect the results, and would report their diet on a regular basis. With that information for the individual the software agent would determine the optimal diet and/or treatments to ameliorate IBD for the individual.

The foregoing has outlined rather broadly several aspects of the present invention in order that the detailed description that follows may be better understood.

### BRIEF DESCRIPTION OF THE DRAWINGS

FIG. 1 is an exploded view of components of a fecal sample container and other components of a fecal sampling kit.

FIG. 2 is an assembled fecal sample container of the components shown in FIG. 1, with a fecal sample outside.

FIG. 3A is a sectional view of the upper part of the assembled fecal sample container taken along a vertical plane with the fecal port and port control in closed position and the lower gate in chamber 106 closed.

FIG. 3B is a sectional view of the upper part of the assembled fecal sample container taken along a vertical plane with the fecal port and port control in closed position and the lower gate in chamber 106 open.

FIG. 3C is a sectional view of the upper part of the assembled fecal sample container taken along a vertical plane with the fecal port and port control in open position and the lower gate in chamber 106 closed.

FIG. 4A is a sectional view of the fecal sample container taken along a vertical plane with the fecal port and port control in open position and the lower gate in chamber 106 closed.

FIG. 4B is a sectional view of the fecal sample container taken along a vertical plane with the fecal port and port control in closed position, a fecal sample in place in chamber 106, and the lower gate in chamber 106 closed.

FIG. 4C is a sectional view of the fecal sample container taken along a vertical plane with the fecal port and port control in closed position, and the lower gate in chamber 106 open, allowing the fecal sample to fall into the tube 101.

FIGS. 5A and 5B are two successive pages of a flow chart showing a method for determining correlations between (independent variables) certain genetic markers, gene expression levels, volatile organic compound levels, and hydrogen and methane levels in the breath (or feces), to both adverse events and particular treatments or diet, for IBD patients. It further shows how to confirm the correlation for an individual with IBD, and messaging that individual to adhere to a recommended diet, and optionally, messaging the individual to report the diet accurately (if the data and analysis indicates it is not reported accurately).

FIGS. 5C and 5D are two successive pages of a flow chart showing a method for determining correlations between (independent variables) certain genetic markers, gene expression levels, volatile organic compound levels, and hydrogen and methane levels in the breath (or feces), to both adverse events and particular treatments or diet, for IBD patients. It further shows how to confirm the correlation for an individual with IBD, and messaging that individual.

FIGS. 6A and 6B show the equations representative of some of the steps in FIGS. 5A and 5B.

FIGS. 6C and 6D show the equations representative of some of the steps in FIGS. 5C and 5D.

FIGS. 7A and 7B are two successive pages of a flow chart showing fecal sample testing for generating preferred gut bacterial composition/microbiome with a low nonpreferred foods and/or low FODMAP diet.

FIGS. 7C and 7D are two successive pages of a flow chart showing a method for determining correlations between (independent variables) certain genetic markers, gene expression levels, volatile organic compound levels, and hydrogen and methane levels in the breath (or feces), to both adverse events and to practicing an unregulated therapy, for IBD patients. It further shows how to confirm the correlation for an individual with IBD, and messaging that individual to adhere to a demonstrably efficacious therapy; and optionally, messaging the individual to adhere to the therapy more strictly (if the data and analysis indicates the therapy should be effective).

For a more complete understanding of the present invention, and the advantages thereof, reference is now made to the following description in conjunction with the accompanying drawings, outlined above.

### DETAILED DESCRIPTION

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in Value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, the term “comprising,” when utilized, means “including, but not necessarily limited to”; it specifically indicates open-ended inclusion or membership in the so-described combination, group, series and the like.

It is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in any appropriately detailed structure.

It is proposed that significant insights into a person's health can be gained by analyzing crowd wisdom, based on aspects of home life, medical narratives, observations on effectiveness of treatments, and generally how diseased subjects operate on a daily basis. The types of insights gained by understanding a subject's social structure, daily routines and cultural background is important in understanding and personalizing that subject's experience and how they cope with chronic conditions or diseases. This type of unstructured data can be collated and analyzed to provide personalized treatment plans for other sufferers to adhere to provide positive changes in their quality of life. Thus, there is a need for processes that will capture a more inclusive set of data and related information from unstructured sources.

This invention is related to systems and methods for capturing crowd wisdom in developing individualized treatment plans. More particularly, the invention is related to a system for data mining crowd sourced structured health related information and unstructured medical narratives and storytelling to identify treatment plans and general techniques that individuals with chronic diseases/symptoms can use to improve their general health and wellbeing. The data mining can be from a wide variety of sources, including the internet, social media, clubs, websites and communities of subjects created for the data mining. The mining can be done using machine learning AI, including deep learning, to find potential treatments.

Embodiments of the invention include systems and methods for capturing crowd wisdom in developing individualized treatment plans. More particularly, the invention is related to a system for data mining crowd sourced structured health related information and unstructured medical narratives and storytelling to identify treatment plans and general techniques that individuals with chronic diseases/symptoms can use to improve their general health and wellbeing. The crowd wisdom is captured by an information management system that has a processing unit that stores a number of software agents. The software agents include a data extraction application that extracts, identifies and links associated processed data from the structured database, the unstructured database and the internet usage database.

Crowd wisdom on disease treatment can be captured from participants in an information management system. Preferably, the participants suffer the disease or condition being investigated for treatment, or are caregivers for the sufferers, with firsthand knowledge of treatment effectiveness. The participants should be screened for obvious bias, such as a financial interest in particular treatments.

The crowd wisdom can be preferably collected from participants using a questionnaire, for example, by sending the questions to their mobile device (such as a smart phone or through a computer). The questions would focus on the specifics of the treatment regime and its application; e.g., the dosage and frequency (if the treatment is a drug, food or other consumable) or how to perform exercise routines and their duration (if they are the treatment). One can also perform data extraction and analysis, using an application to data mine the structured health related information such as medical records, pharmacy records, unstructured medical narratives such as storytelling, and internet usage data to identify treatment plans and general techniques that individuals with diseases/symptoms use. The subjects can also be individually monitored to try to determine the source of symptom relief, if they are unaware of why their symptoms might periodically improve.

The crowd wisdom from the foregoing sources is sent to a processing unit with a number of software agents and data extraction applications, including inference engines, that it uses to identify and associate relevant information in each data source and to correlate and link the relevant information identified in each data source to build a searchable combined information system, that is communicated to a web portal or platform. The combined information system on the web portal or platform is searchable and downloadable to a user through a mobile device controlled by, e.g., an application or software agent which instructs and configures communication between the combined information system and the mobile device.

In one embodiment of the information management system, the unstructured data will include a daily logging of users daily symptoms, general wellbeing, habits and routines. The collective wisdom of thousands of participants captured within these daily medical narratives and personal experiences will be processed using various algorithmic based data extraction applications. For example, common themes of symptoms, daily routines, and treatments plans (medically recommended and user determined alternative health treatments) will be extracted and correlated using a variety of software analytic engines such as natural language processing, inference engines, and by following Markov patterns. The key is to dissect these conversations and stories for patterns of commonality as well as unique patterns that offer insights into the best practices for achieving the best outcomes. The analytic engines will extract information from the medical narratives, match and compare the information extracted from participants and assess similarities and differences. The similarities and differences will be collated and analyzed to extract common information, on what are widely agreed to be effective treatments by participants. The more widely effective treatments would be first preference for clinical trials or participant testing.

The invention also includes rigorous testing of proposed treatments by first determining a set of IBD indicators which can be objectively measured, and then performing the indicator measurements in subjects reporting symptom relief from a treatment to determine if the indicators correlate with the expected pattern for symptom relief. The objective indicators include measurement of hydrogen and/or methane off gassing of an IBD subject's breath (or from feces). Embodiments of the invention are related to a system providing a hydrogen and/or methane sensor device and a wireless platform in communication with the sensor device to periodically analyze the hydrogen and/or methane off gassing of an IBD subject's breath (or from feces) and correlating the levels of hydrogen and/or methane with symptoms or Events. For preferably determining levels of hydrogen and/or methane, subjects periodically measure methane and hydrogen in the breath, using a wireless device which sends the results to a server. See e.g., US Publ'n No. 20180271404 (disclosing a methane and hydrogen sensor for breath, to integrate with a smartphone or other device). Subjects report their symptoms and food intake, preferably using a wireless device which sends the results to the server. One preferred method for measuring the hydrogen/methane in a fecal sample, is to include a tube running from upper chamber 104 and detachably connecting to the breath sampler device (as described in U.S. Publ'n No. 20180271404), which then measures the levels of hydrogen/methane in the fecal sample from the gas level in chamber 104.

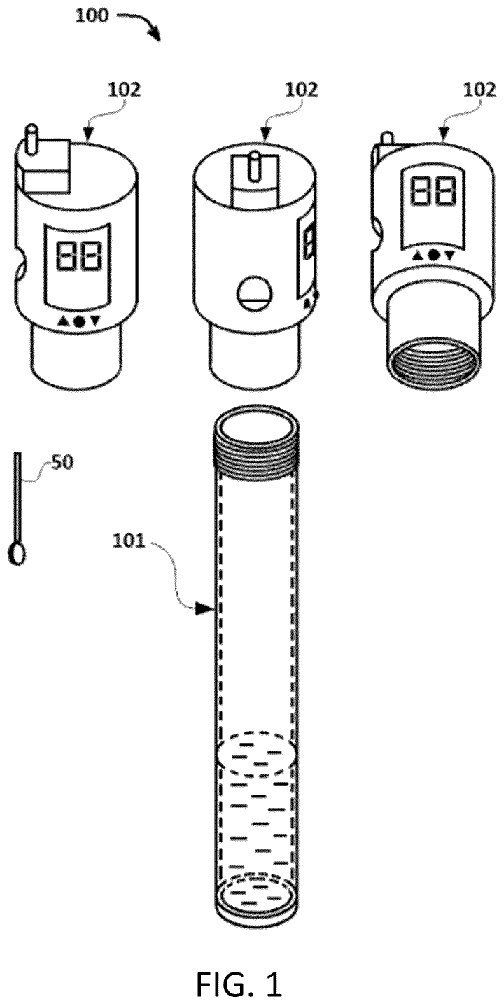

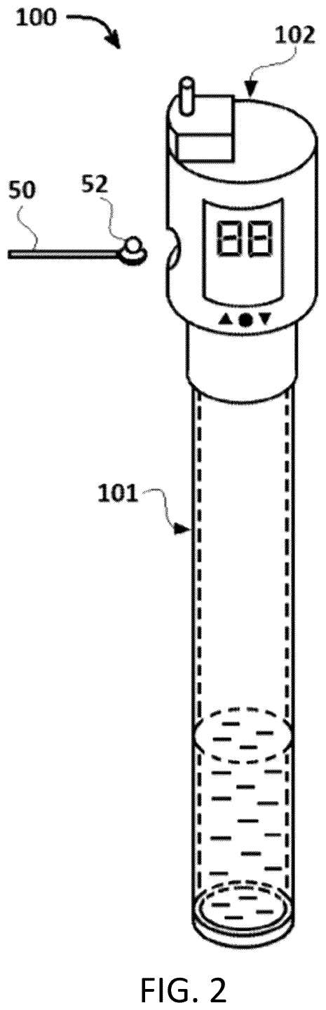

Referring to FIG. 1, it shows a fecal sampling kit 100 with a spatula 50 and a fecal sample container 101. Spatula 50 can be used by the subject to scoop a small measured amount of fecal matter into chamber 106. As noted in the Summary, hydrogen and/or methane is preferentially measured from the subject's breath, using a device as described in U.S. Publ'n No. 20180271404. But it could also be measured from a fecal sample, where a relatively standard volume of fecal material 52 is placed in chamber 106, and the off gassed hydrogen or methane is measured. When measured from the fecal sample, the same device for measuring hydrogen and/or methane from the subject's breath can be attached (using a tube, for example) to a port in the upper chamber 104 of fecal sample container 101.

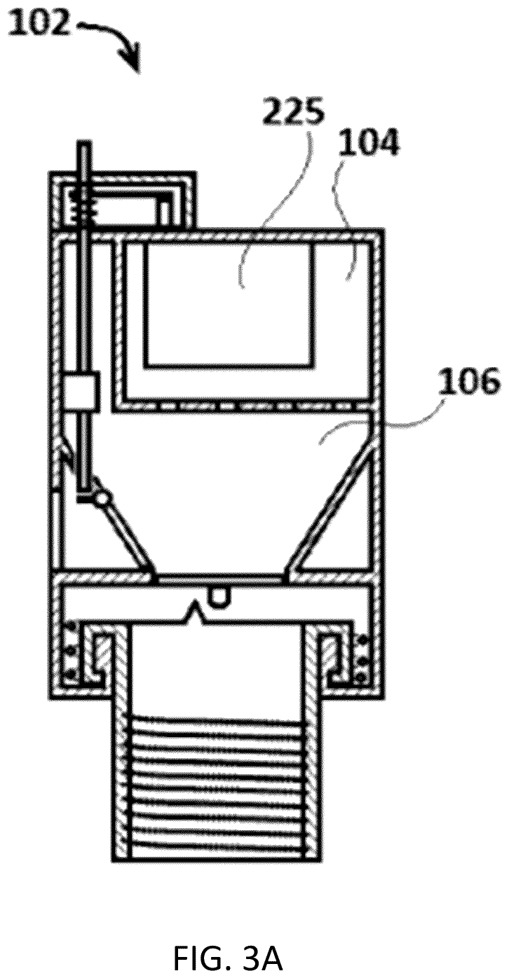

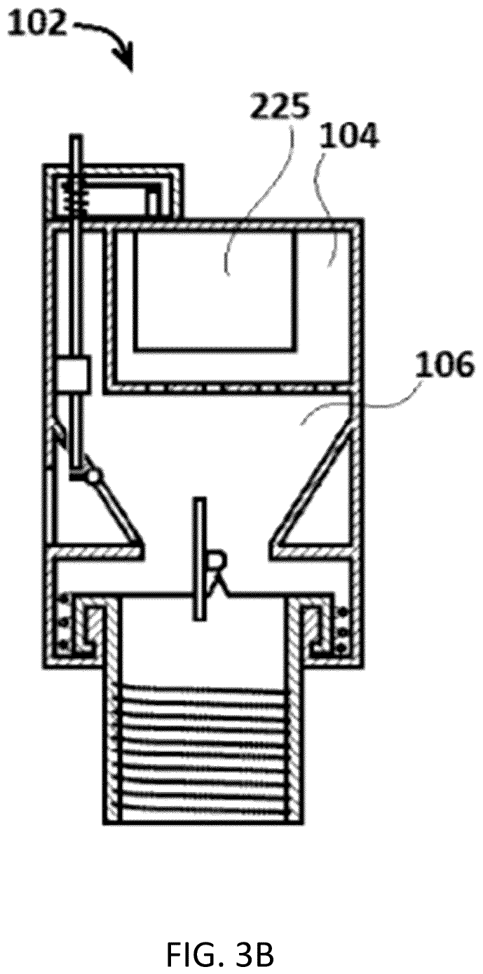

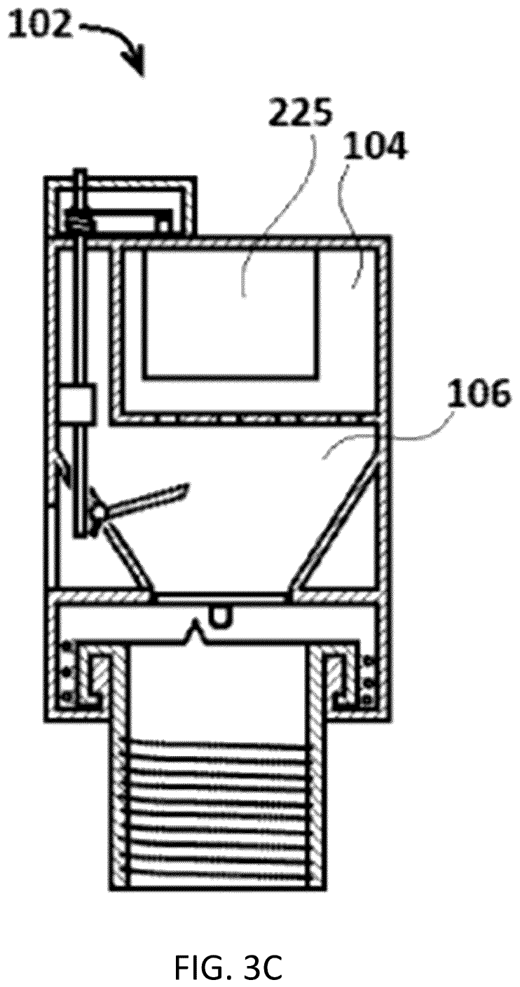

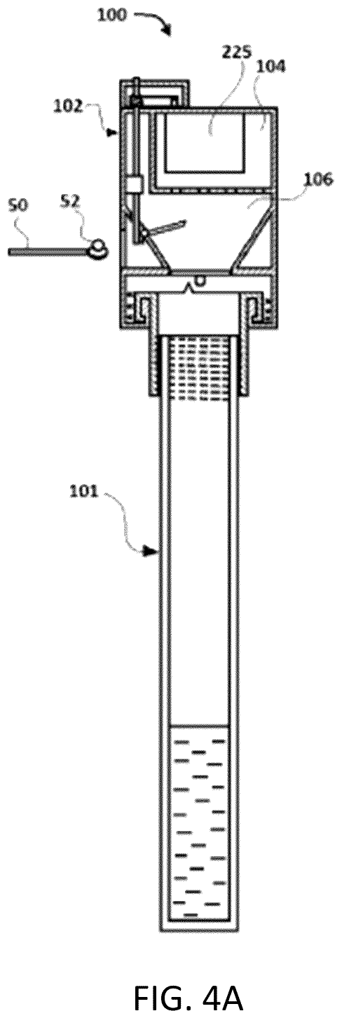

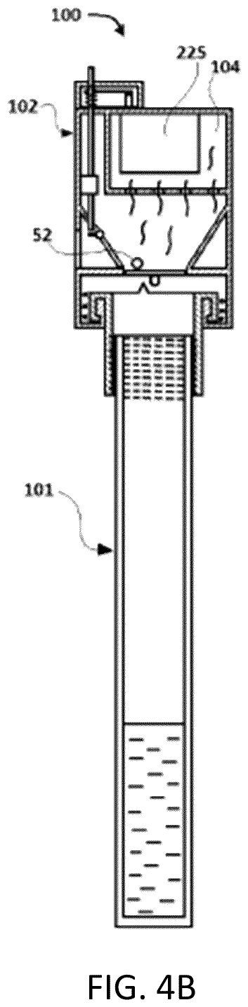

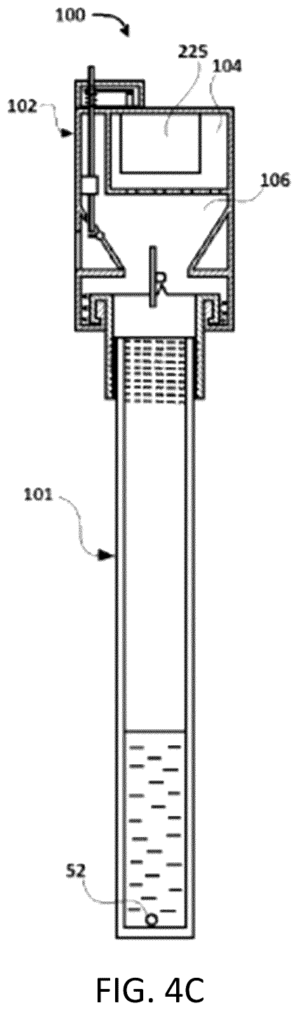

Referring to FIGS. 3A to 4C, fecal sample container 101 has a threaded cap 102 with two chambers, 104, 106, each of which is sealed but can be accessed by spatula, 50, carrying a solid, like fecal sample 52. Upper chamber 104 is sealed from the environment and capable of collecting gases from a sample 52 in the cap's lower chamber 106. The lower trap door of the lower chamber 106 is opened by e.g., twisting the cap 102 to allow sample 52 therein to fall into container 101. An automatic timer 225 in cap 102 is activated by a sensor, which detects when the port on the side of chamber 106 is opened for the sample to enter. The automatic timer 225 is preferably set to limit the gas measurement by a gas sensor (now shown) for a specific period of time (e.g., 10 seconds, 20 seconds). Several off gassed hydrogen/methane measurements are taken in succession and stored as individual values specific for a particular fecal sample of the individual subject. In one embodiment, all measurement values and related subject information for a specific fecal sample are recorded and then transferred to a data processor, preferably wirelessly, such as by Bluetooth to a mobile phone or other wireless device. One preferred method for measuring the hydrogen/methane in a fecal sample, is to include a tube running from upper chamber 104 and detachably connecting to the breath sampler device (as described in U.S. Publ'n No. 20180271404), which then measures the levels of hydrogen/methane in the fecal sample from the gas level in chamber 104.

After cap 102 is manipulated to open the lower chamber 106, the lower trap door in chamber 106 opens and fecal sample 52 falls into container 101. Cap 102 is twisted again to seal sample 52 in container 101. Container 101 is then shipped for fecal sample analysis and/or optionally methane and hydrogen gas analysis, or, optionally VOC analysis, of the gas collected in the upper chamber 104. Optionally the fecal sampling kit 100 may also contain a sealable impermeable pre-addressed bag that the fecal sample container 101 is placed into for mailing to a laboratory for analysis. The fecal sample 52 analysis can be for levels of the compounds in Tables A and B above, genetic markers associated with IBD, levels of gene expression correlating with IBD, and the bacterial composition of the sample.

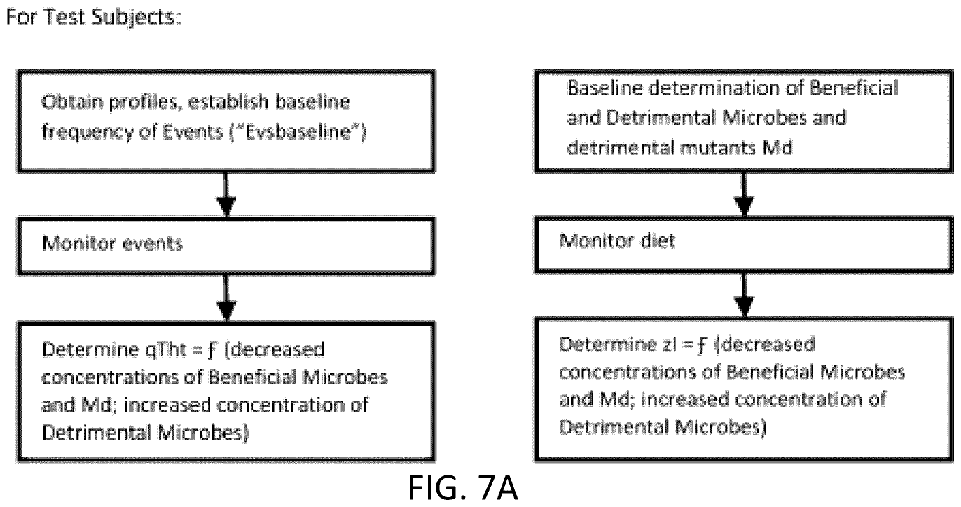

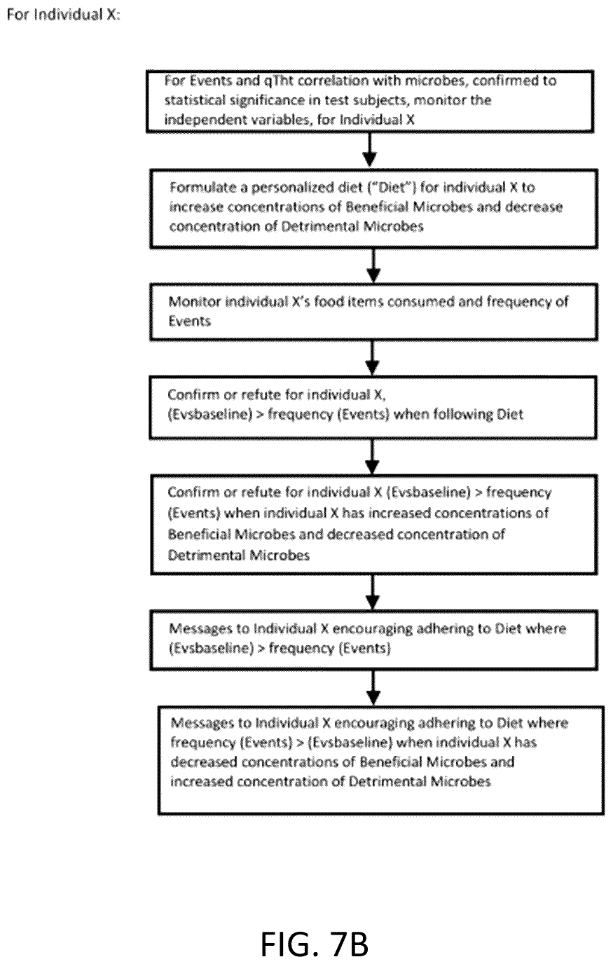

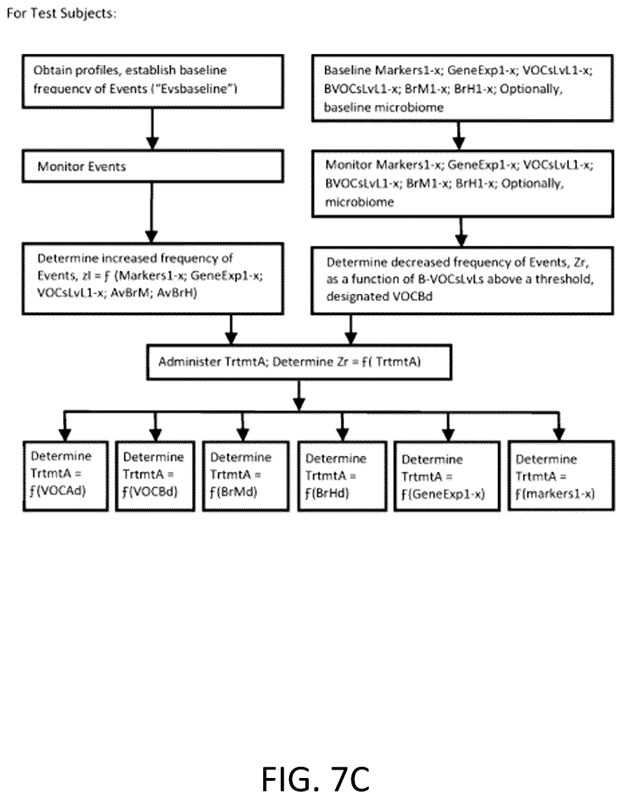

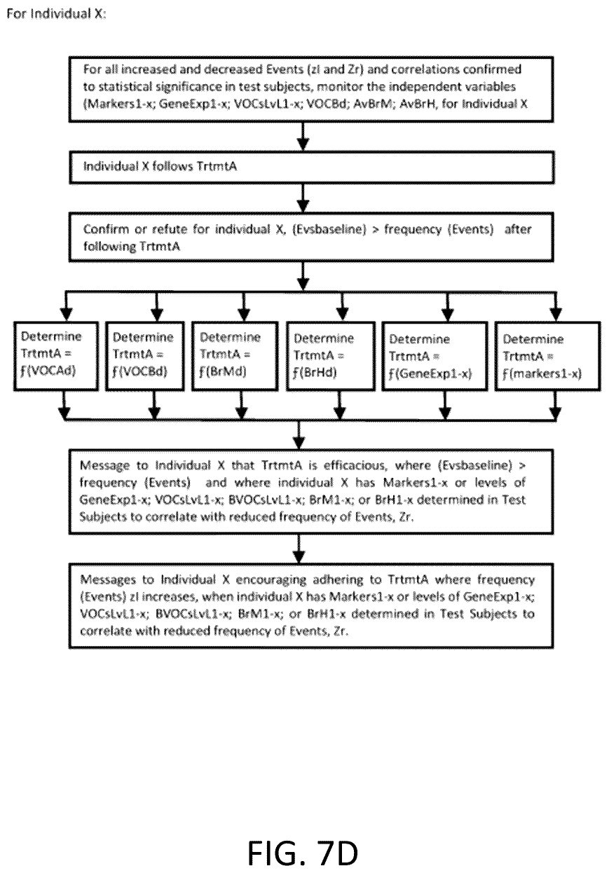

Following finding of potential treatments through crowd-sourcing, public documents or other methods, these treatments are tested in a group of test subjects. FIGS. 7C-7D outline the testing and verification of effective treatments in a group of test subjects for sufferers which exhibit symptoms as Events; for example, for those with inflammatory bowel disease (IBD), Events would include frequent diarrhea, high frequency flatulence, and high frequency bowel movements. The baseline frequency of Events (“Evsbaseline”) is established for each test subject before starting treatment. In FIGS. 7C-7D, where the steps are shown in flow chart form with limited explanation, the following steps 1 to 22 are a comprehensive explanation of the steps in the FIGS. 7C-7D process. Events can be determined directly from subject's reporting, or from external sources such as medical, medical insurance or pharmacy records or reports.

A personalized database for subjects is created by capturing data from multiple sources, including medical records such as their medical history, laboratory data, diagnoses, treatment plans, and family medical history. Another example of such data is data measured by one or more biodata or sensor devices. Biodata can be collected by devices equipped with the necessary software to process the data generated by the device and to communicate the collected data to the personalized database and from there, to a processor. Examples of such biodata-collecting devices include glucometers, hydrogen or methane sensors, temperature sensors, heart rate monitors, blood pressure monitors, and activity sensors with tri-axis or multi-axis accelerometer chips.

The personalized database will also include input from extensive questionnaires and individualized insights gained by understanding the subject's social structure, daily routines and cultural background to assist in understanding how that person copes with a chronic condition or symptoms on a daily basis. This type of data is vital to finding personalized treatment plans that a person will adhere to and that will provide positive changes in a person's quality of life. Although people share the same disease or the same chronic symptomology, their ability to navigate through the symptoms often will include personal adjustments to their daily routines. Many people facing chronic diseases and associated uncomfortable or embarrassing symptoms will try alternative approaches that they are unwilling to share with their physicians. Thus, a great deal of relevant information does not appear in their medical records. Often these alternative approaches will include herbal remedies, supplements, acupuncture or acupressure, reflexology, relaxation techniques, or exercise regimes such as yoga or stretching.

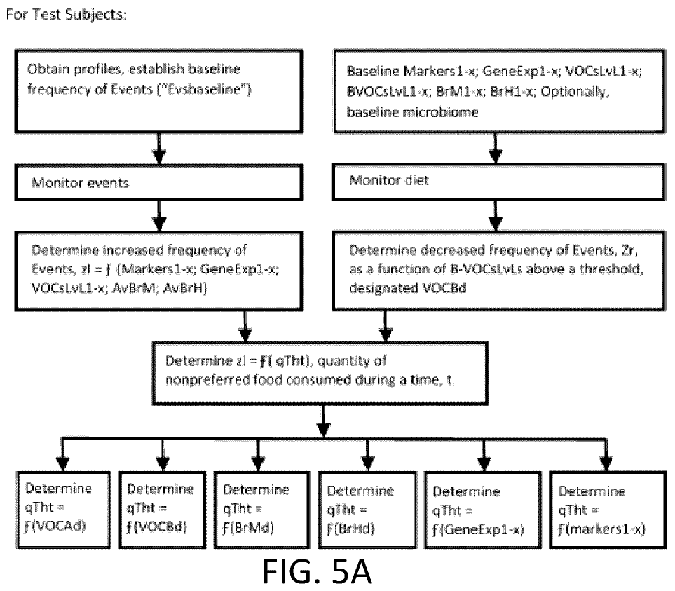

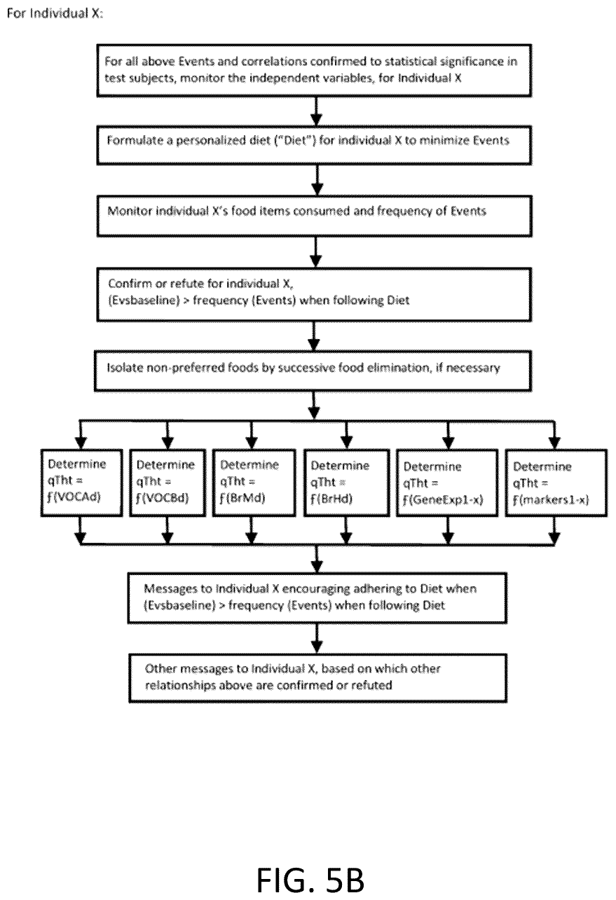

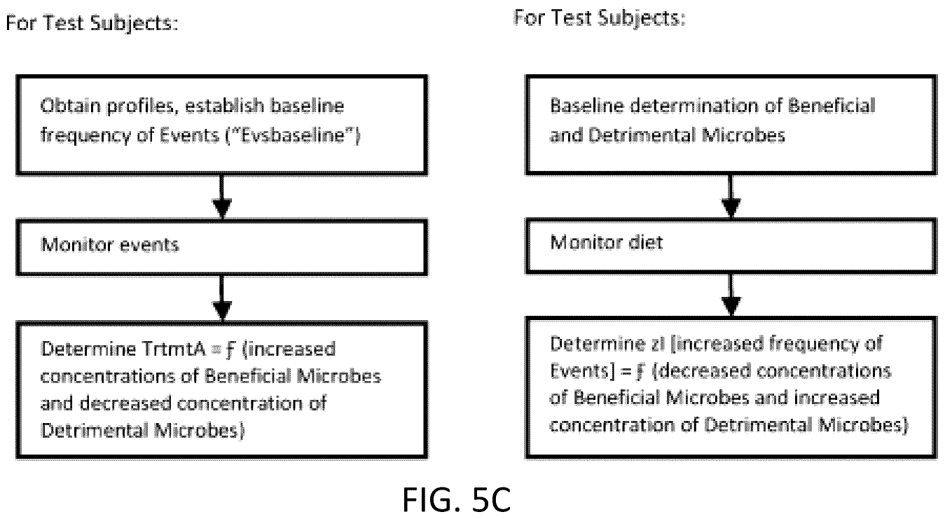

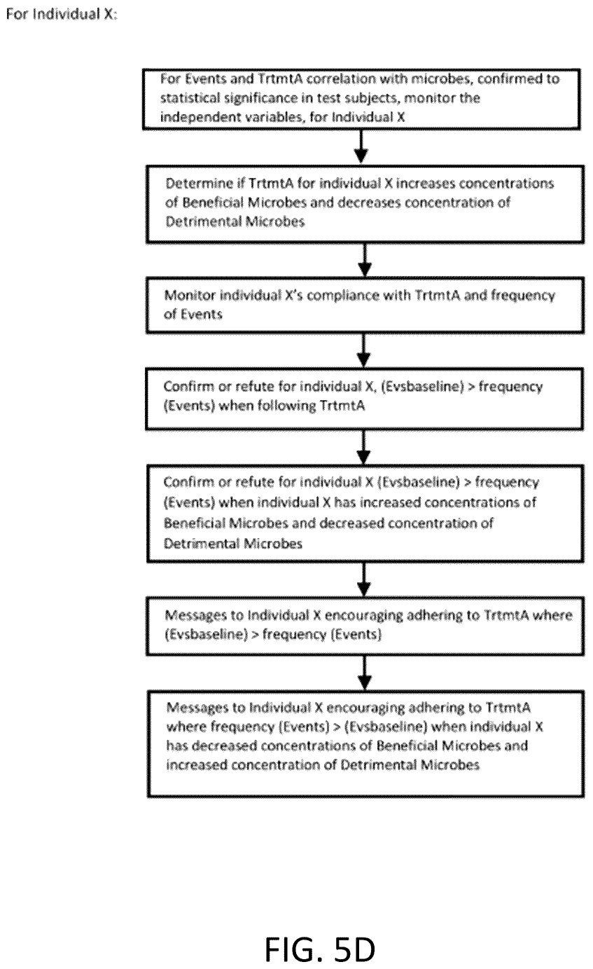

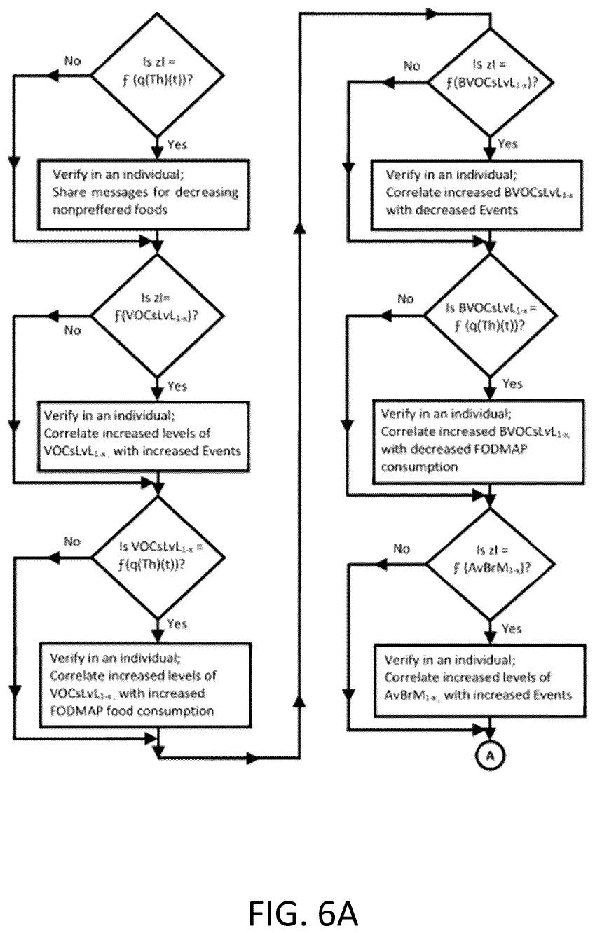

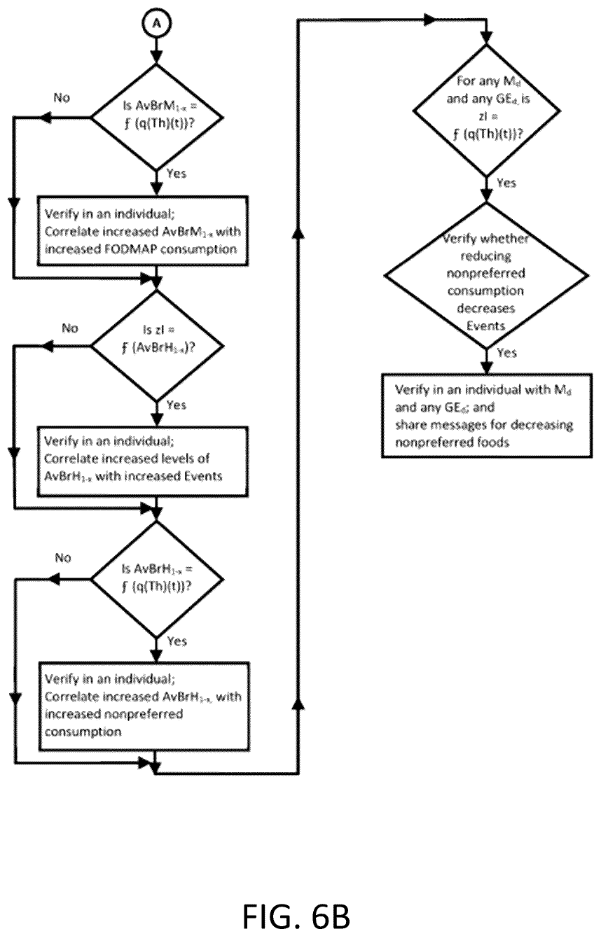

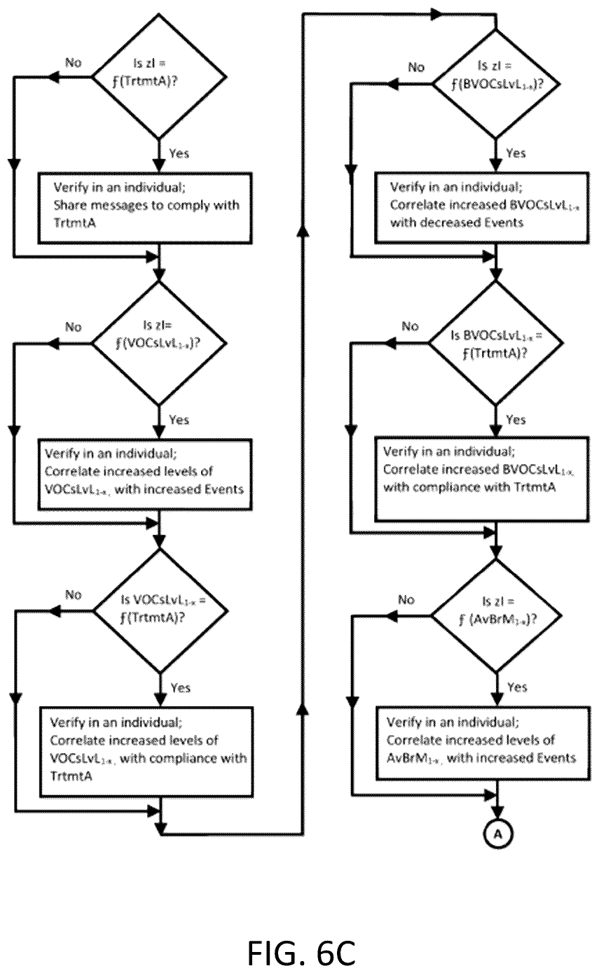

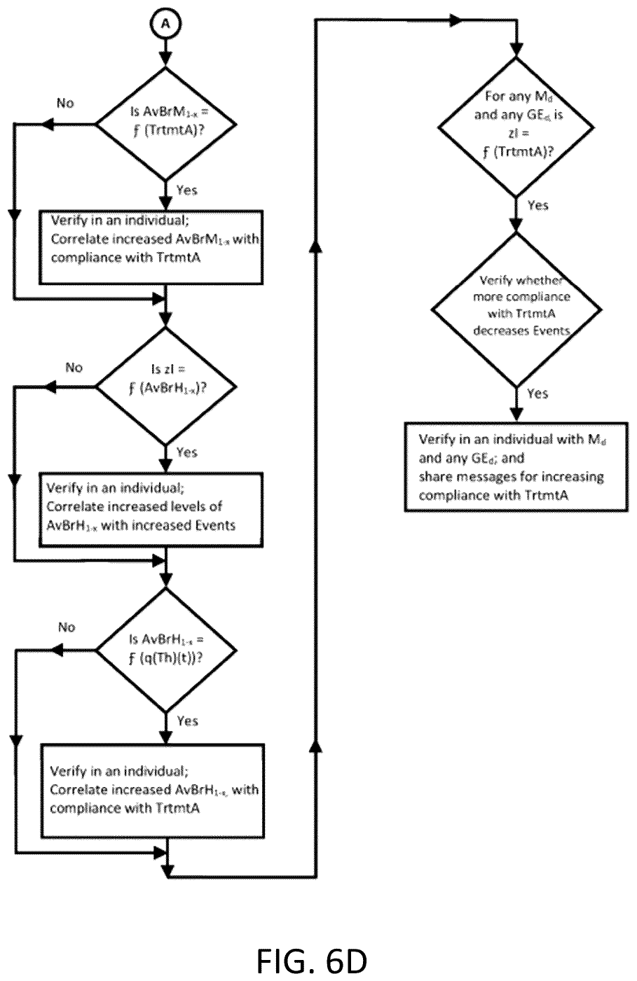

A processor stores a number of software applications and agents executable by the processing unit. The software applications and agents include a data extraction and analysis application that extracts, identifies and links associated processed data acquired from various sources. The data extraction applications include inference engines and other algorithmically based applications used to identify and correlate relevant information in a personalized database and external data sources. The processor, through agents and applications, is capable of performing all the operations described in the flowcharts in FIGS. 5A-5D, 6A-6D, and/or 7A-7D.

In one embodiment, the processor can include a processing unit having a multitude of interrelated elements. Embodiments of the processing unit can be implemented to some extent as software modules installed and running on one or more processing systems, such as servers, workstations, tablet computers, PCs, and so on. The processor generally includes a knowledge module that derives further knowledge or informational data from existing knowledge using inference, analysis, crowd sourced wisdom and continuous monitoring data from a personalized database of the subject.

Thus, the knowledge module is a “care” analysis engine that stores its data in the data repository. The data repository can include one or more databases that communicate with the knowledge module. The knowledge module can also receive data from an external data sourced database. The external sourced database may include data from various sources, such as laboratories, insurance companies, hospitals/clinics. media companies, 24/7 call centers/caregivers, account administrators, and other sources. The data from the external database can be extracted and transferred to the knowledge module using dynamic APIs.

The processor processes information accessed and derived by the knowledge module to determine personalized clinical and nutritional decision analytics for subjects or individual system participants. The processor may include one or more algorithms that provide both content and personalized rules to provide feedback to the user in real time. For example, the processor may include code for predicting trends based upon the subject's personalized health profile and preferences. The information acquired from a subject's personalized data input by the subject and data derived from an analysis of subject samples may be stored in the data repository and accessed by the processor.

The data extraction applications on the processor identify, analyze and correlate relevant information in the personalized database and data continuously gathered, including hydrogen and methane levels (sourced from breath or fecal off-gas), the genetic marker and/or gene expression analysis, the analysis of the VOCs in patient feces; and, as input by the subject: the diet and foods consumed and the adverse events (symptoms of IBD). See FIGS. 5A-6B. The data continuously gathered can also include gut bacterial composition. See FIGS. 7A, 7B.

The processor includes applications/agents which, based on the analysis of the personalized database and the data continuously gathered, send messages to subjects regarding food consumption; particularly, how to better conform to a low FODMAP or other preferred diet. The processor may also message daily meal, an activity plan or exercise plan for the subject, recommendation to contacting a coach or health guide (a Health Sherpa), nutritional guidance, subject reports and predictions, subject health profiles and videos or chat sessions.

Typically, the processor will include code for predicting trends based upon the individual subject's personalized health profile and preferences. For example, the rules may pertain to a daily meal or activity plan for the participant based on personal preferences, a matching of the subject to one or more health consultants, nutrition guidance, exercise routines, general health predictions and alerts, trends and improvements in the subject's health profile and video/chat sessions. Various parameters are considered in determining recommendations, educational messages, and directives to the subject. The processor analyzes and correlates the relevant data to determine useful information for the specific subject and transmits that information to the subject, including information relating to nutrition, exercise advice and treatment. Access privileges to any processor information may also be driven rules based on health care privacy regulations and laws.

A portal on the processor, which is preferably web-based, can be provided by the processor. The portal can provide interfaces for reporting and displaying the data analyzed by the processor, and including information and recommendations for the subject or the health care personnel, or messaging for the subject. Portal access is controlled through established access privileges.

To use the care management system of the invention, the subject uses fecal sampling kit 100 and preferably, also a breath sampling unit. The subject will collect a fecal sample 52 using a spatula 50 and place it into fecal sample container 101. The subject will then either follow the procedure to use the cap system to measure hydrogen and methane from the sample, or use the breath sensor in the alternative. The gas measurements are then communicated to the processor, and the fecal sample container is sealed and sent for genetic and VOC analysis, and optionally, for bacterial composition.

The processor's software agents analyze and correlate all analysis including those input to the personalized database, to enhance the ability of the care management system to provide updated and relevant guidance, through messaging to the subject. For example, the software agents will correlate changes in incidence of symptomatic events with levels of methane and hydrogen, VOCs, genetic markers and gene expression, as well as diet, and optionally, the feces bacterial composition. The specifics of these correlations and analysis are illustrated in FIGS. 5A-7B.

Another use for the analyses set forth in FIGS. 5A-7B, other than optimizing diet as illustrated, is to determine effectiveness and dosage of therapeutic drugs on the subject. The same analysis can be used as for diet optimization, to find drugs and dosages which effectively reduce symptomatic events (“Events” in FIGS. 5A-7B).

Referring to FIGS. 5A, 5B, where the steps are shown in flow chart form with limited explanation, the following steps 1 to 23 are a comprehensive explanation of the steps in the FIGS. 5A, 5B process.

1) Obtain details of digestive health of each test subject: health profiles, and past and present digestive ailments i.e.: whether suffering from IBS, IBD, frequent diarrhea, high frequency flatulence, high frequency bowel movements (“Events” or “symptoms”), and also the baseline frequency of Events (“Evsbaseline”) and prepare test subjects on how to report (“Events” or “symptoms”);

2) Perform baseline fecal sample collection for test subjects to initially determine: (i) DNA and/or RNA markers for wild type and mutant beneficial or detrimental bacterial strains in the gut microbiome (“Markers1-x”); (ii) gene expression of any candidate genes (from the individual or the microbiome bacteria) whose expression is elevated or decreased in the test subjects from normal (“GeneExp1-x”); (iii) levels of volatile organic compounds in Table A (“VOCsLvL1-x”) and in Table B (“B-VOCsLVL1-x”) associated with test subjects where the VOCsLVL and B-VOCsLVL can be measured from gas associated with the fecal sample; and (iv) determine from a breath sensor carried by test subjects the methane (“BrM”) and hydrogen levels (“BrH”) in each subjects' breath (or feces), where 1-x indicates a reading at a different time for methane and hydrogen levels, which can be transmitted to a server;

3) Monitor treatment and/or diet of each test subject using a wireless device which allows input of all information about the treatment (TrtmtA) and food items and quantity consumed; with recommendations on screen for low FODMAP foods, and preferably showing carbohydrate content and glycemic Index, fat and protein content, gluten levels, for each meal or for each food item;

4) Monitor each test subject's frequency of Events using the wireless device, which allows their entry and category;

5) Collect fecal samples at intervals for each test subject: and monitor changes in Markers1-x; GeneExp1-x; VOCsLvL1-x; and B-VOCsLvL1-x; Optionally: BrM or BrH, preferably, determine the average of the readings (“AvBrM” and “AvBrH”) during each collection period; (Optionally, monitor the methane (“M1-x”) and hydrogen levels (“H1-x”) in each subjects' fecal sample and use those measures for AvBrM and AvBrH, instead of measuring them from the subject's breath.)

6) Use a software agent to determine dependency of increased frequency of Events, zl (as the dependent variable), with the independent variables being: Markers1-x; GeneExp1-x; VOCsLvL1-x; AvBrM; AvBrH; wherein all independent variables are determined across all test subjects; using a software agent which (i) determines correlation of zl with each independent variable using a univariate hypothesis test, where the null hypothesis is “the presence of this marker, or this level of gene expression or greater, or this level of volatile organic compounds or greater, or these levels of hydrogen and methane or greater, are not associated with zl”; where such markers are designated Md, such gene expression levels are designated GEd, such levels of organic compounds are designated VOCAd; such average levels of methane are designated; BrMd, and such average levels of hydrogen are designated BrHd (ii) performs a multivariate regression model of all possible combinations of the independent variables Markers1-x; GeneExp1-x; and VOCsLvL1-x, AvBrM; AvBrH with substantially the same null hypothesis as in step (i) but where the word “or” is “and”; to represent the combination of independent variables modeled, where the following formula represents this multivariate regression model:

$$z l = ( Markets 1 - x ; GeneExp 1 - x ; VOCsLvL 1 - x ; AvBrM ; AvBrH )$$

Determine the independent variables and combinations thereof where the dependency of increased frequency of Events, zl, is established at a confidence interval (CI) of at least 95% for the appropriate null hypothesis noted above in steps (i) and (ii); for markers Md, for gene expression levels GEd, for levels of organic compounds VOCAd; for levels of methane AvBrM; and for levels of hydrogen AvBrH.

7) Use a software agent to determine dependency of increased frequency of Events, zl (as the dependent variable), with the dependent variable being TrtmtA (as represented by the formula zl=(TrtmtA)) and/or a diet with consumption of a quantity, q, of FODMAP or other specified (non-preferred) foods above a threshold, Th, consumed in a period of time, t, as represented by the formula: zl=(q (Th) (t)), where such consumption is designated “qTht”; where the dependency of increased frequency of Events, zl, is established at a confidence interval (CI) of at least 95% (where the null hypothesis is: “failure to follow TrtmtA” or “consumption of a quantity, q, of FODMAP or other non-preferred foods above a threshold, Th, consumed in a period of time, t, are not associated with increased frequency of Events, z,” respectively;

8) Use a software agent to determine dependency of decreased frequency of Events (as the dependent variable, Zr), with the independent variable being a level of volatile organic compounds in Table B (“B-VOCsLVL1-x”) above a threshold associated with normal subjects or remission (see specification), where the B-VOCsLVL can be measured from gas associated with the fecal sample, and where such dependency is established at a confidence interval (CI) of at least 95% (where the null hypothesis is: “Levels of B-VOCsLvLs above this threshold are not associated with decreased frequency of Events, Zr”; where such levels are designated VOCBd), where the following formula represents this step 8: Zr=(BVOCsLVL1-x)

9) Use a software agent to determine correlation between TrtmtA or consumption of qTht and levels of VOCBd (above or below the threshold, respectively): TrtmtA= (VOCBd); qTht=(VOCBd); at a confidence interval (CI) of at least 95%, by using as a null hypothesis: “TrtmtA does not correlate with levels of VOCBd above the threshold” or “consumption of qTht does not correlate with levels of VOCBd below the threshold.”

10) Use a software agent to determine correlation between TrtmtA or consumption of qTht and levels of VOCAd (below or above the threshold, respectively): TrtmtA=(VOCAd); qTht=(VOCAd); at a confidence interval (CI) of at least 95%, by using as a null hypothesis: “TrtmtA does not correlate with levels of VOCAd below the threshold” or “consumption primarily of qTht does not correlate with levels of VOCAd above the threshold.”

11) Use a software agent to determine correlation between TrtmtA or consumption of qTht and levels of methane BrMd (below or above the threshold, respectively): TrtmtA=(BrMd); qTht=(BrMd); at a confidence interval (CI) of at least 95%, by using as a null hypothesis: “TrtmtA does not correlate with levels of BrMd below the threshold” or “consumption primarily of qTht does not correlate with levels of BrMd above the threshold.”

12) Use a software agent to determine correlation between TrtmtA or consumption of qTht and levels of hydrogen BrHd (below or above the threshold, respectively): TrtmtA=(BrHd); qTht=(BrHd); at a confidence interval (CI) of at least 95%, by using as a null hypothesis: “TrtmtA does not correlate with levels of BrHd below the threshold” or “consumption primarily of qTht does not correlate with levels of BrHd above the threshold.”

13) Based on an individual X's profile including dietary restrictions, and the diet determined in steps 7 and 9 to minimize Events, determine if TrtmtA is appropriate and formulate a personalized diet (“Diet”) for individual X to minimize Events in view of the individual X profile and dietary restrictions, and provide the steps and procedures for TrtmtA and/or the Diet to individual X on the wireless device;

14) Monitor individual X's compliance with Trtmt A and/or food items consumed and frequency of Events, based on entries in individual X's wireless device;

15) Confirm or refute for individual X correlation of Trtmt A and/or the Diet with decreased frequency of Events (“Zr”) as compared with Evsbaseline; i.e., individual X following the Trtmt A and/or Diet results in (Evsbaseline)>frequency (Events);

16) Confirm or refute for individual X, a decreased frequency of Events (Zr) as compared with Evsbaseline; for each of: gene expression levels<GEd, VOCsLvL<VOCAd, and B-VOCsLvL>VOCBd; AvBrM<BrMd; AvBrH<BrHd; and an increased frequency of Events (zl) as compared with Evsbaseline (i.e., frequency (Events)>(Evsbaseline)) with the presence of markers Md in the sample;

17) Confirm or refute for individual X that Trtmt A and/or consumption of qTht correlates with levels of VOCAd (below or above the threshold, respectively) (Correlation A);

18) Confirm or refute for individual X that Trtmt A and/or consumption of qTht correlates with levels of VOCBd (above or below the threshold, respectively) (Correlation B);

19) Confirm or refute for individual X that Trtmt A and/or consumption of qTht correlates with levels of BrMd (below or above the threshold, respectively) (Correlation C);

20) Confirm or refute for individual X that Trtmt A and/or consumption of qTht correlates with levels of BrHd (below or above the threshold, respectively) (Correlation D);

21) If individual X shows a decreased frequency of Events (Zr) where any of the following are true: VOCsLvL<VOCAd; B-VOCsLvL>VOCBd; AvBrM<BrMd; AvBrH<BrHd; and markers Md are absent; and if any of Correlations A through D are established for individual X, but individual X does not show decreased frequency of Events by following Trtmt A and/or the Diet, message individual X to carefully monitor compliance with TrtmtA and do one or more of: enter all food consumed, accurately report of food intake or do not enter foods erroneously; and if individual X continues to not show decreased frequency of Events by following the Diet, send messages to individual X to sequentially eliminate particular foods typically consumed until either all foods consumed are eliminated and deemed not causative, or foods associated with the failure to decrease frequency of Events zl are identified (i.e., which foods fail to alleviate symptoms); and

22) If individual X shows decreased frequency of Events when following TrtmtA and/or the Diet, and shows increased frequency of Events when not following TrtmtA and/or the Diet: message individual X about (i) TrtmtA and/or the Diet and/or the importance of consistently following the Diet and (ii) specifying in the messages how to conform individual X's food intake to TrtmtA and/or the Diet, based on the food intake reported by individual X during periods when there was increased frequency of Events (see flow-chart B examples).

The steps 1-22 above and FIGS. 5A and 5B provides a rigorous analysis of a number of data points from test subjects, with the objective to provide an optimized diet for an individual with IBD. It shows further, how to construct messages for the individual to adhere to or optimize the diet to reduce symptoms of IBD.

The steps 1-22 above and FIGS. 7C and 7D provides a rigorous analysis of a number of data points from test subjects, with the objective to provide an effective treatment for an individual. It shows further, how to construct messages for the individual to adhere to or optimize the diet to reduce symptoms of IBD.

FIGS. 6A-6D summarize steps 1-22 in equation form.

Another set of data points can also be analyzed and then applied to an individual, in addition to those in FIGS. 5A and 5B or FIGS. 7C and 7D and steps 1-22 above; as shown in FIGS. 6A-6D. For the steps in FIGS. 5C and 5D and FIGS. 7A and 7B (and the steps in la et seq below) it is assumed that the correlation in step 7 above, i.e., zl=(TrtmtA) or zl=(q(Th)(t)), had been established in test subjects.

These data points relate to the composition of the gut's bacteria, also known as the microbiome. The full explanation of the flow chart in FIGS. 7A and 7B is as follows in steps 1a to 9a.

1a) Obtain details of digestive health of each test subject: health profiles, and past and present digestive ailments i.e.: whether suffering from IBS, IBD, frequent diarrhea, high frequency flatulence, high frequency bowel movements (“Events” or “symptoms”), and also the baseline frequency of Events, (“Evsbaseline”);

2a) Perform baseline fecal sample collection for test subjects to initially determine: (i) concentrations of Bifidobacteria, Lactobacillus, Faecalibacterium prausnitzii and Propionibacteriaceae (“Beneficial Microbes”); (ii) concentrations of Bacteroides fragilis, Ruminococcaceae and Clostridium (“Detrimental Microbes”); and (iii) presence of any mutants of Beneficial Microbes or Detrimental Microbes (based on presence of any Markers1−x) which are associated with Zr (referred to as “Mutants” and including “Md”).

3a) Monitor compliance diet of each test subject using a wireless device which allows input of steps relating to TrtmtA and/or all food items and quantity consumed; with recommendations on screen for how to do so, and/or recommendations for low FODMAP foods, and preferably showing carbohydrate content and glycemic Index, fat and protein content, gluten levels, for each meal or for each food item;

4a) Collect fecal samples at intervals for each test subject: and monitor concentrations of Beneficial Microbes and Detrimental Microbes and Mutants, during each collection period;

5a) Use a software agent to determine correlation of TrtmtA and/or qTht with decreased concentrations of Beneficial Microbes and increased concentration of Detrimental Microbes and absence of Mutants; and using a univariate hypothesis test, where the null hypothesis is “TrtmtA and/or qTht is not associated with increased concentrations of Beneficial Microbes and decreased concentration of Detrimental Microbes and absence of Mutants;” at a confidence interval (CI) of at least 95% for the null hypothesis;

6a) Use a software agent to determine correlation of increased frequency of events, zl, with decreased concentrations of Beneficial Microbes and increased concentration of Detrimental Microbes and/or presence of Mutants.

7a) If the correlation in step 5a or 6a is established at a confidence interval (CI) of at least 95% for the null hypothesis, confirm or refute for individual X the correlation between TrtmtA and/or qTht and/or zl with decreased concentrations of Beneficial Microbes and increased concentration of Detrimental Microbes and presence of Mutants;

8a) if the correlation in step 5a is established for individual X, but individual X reports compliance with TrtmtA and/or consuming only low FODMAP or other recommended foods and fecal samples do not show increased concentrations of Beneficial Microbes and decreased concentration of Detrimental Microbes and absence of Mutants; send messages to individual X to accurately report steps taken in TrtmtA, and/or sequentially eliminate particular foods typically consumed until either all foods consumed are eliminated and deemed not causative, or foods associated with the failure to increase concentrations of Beneficial Microbes and decrease concentration of Detrimental Microbes reduce Mutants occurred or are identified; and

9a) If individual X shows increased concentrations of Beneficial Microbes and decreased concentration of Detrimental Microbes and/or absence of Mutants when following TrtmtA and/or a low FODMAP or other recommended diet, and shows the opposite when not following TrtmtA and/or a low FODMAP or other recommended diet: message individual X about (i) the importance of consistently following TrtmtA and/or the low FODMAP or other recommended diet and (ii) specifying in the messages how to conform individual X's activities to TrtmtA and/or food intake to the low FODMAP or other recommended diet, based on the information and food intake reported by individual X.

Essentially the same systems as above and in FIGS. 5A-5B, 6A-6B, and 7A-7B could also be used to identify supplements, prescription drugs, exercise or manipulation regimes, or other treatment methods—instead of or in addition to relying on consuming a low FODMAP diet to reduce Events. To find such other treatment methods, the same steps 1-22 and 1a to 9a above (as shown in the flow charts in FIGS. 5A, 5B and 7A, 7B, respectively) can be performed, but with the “other treatment method” being tested substituted for the “low FODMAP diet.” For example, in steps 3 or 3a above, where the treatment tested was a drug, one would monitor each test subject using a wireless device which allows input of the drug dosage consumed. In steps 7 and 5a, one would substitute the drug dosage consumed as the dependent variable. For a drug dosage determined in steps 7 and 8 to minimize Events, that drug dosage can be tested by an individual (“individual X”) to determine if it is effective for the individual in reducing Events. The relationship between the drug consumption and the various indicators in step 16, i.e., “gene expression levels<GEd, VOCsLvL<VOCAd, and B-VOCsLvL>VOCBd; AvBrM<BrMd; AvBrH<BrHd” or between the drug consumption and increased concentrations of Beneficial Microbes and decreased concentration of Detrimental Microbes and Mutants in step 5a, can also be determined as additional verification that the drug is effective for reducing Events. A supplement would be tested and verified in the same manner, as could any type of exercise or manipulation regimes, or other treatment methods.

Where the drug, supplement or other treatment method was shown to reduce Events for individual X, they could be messaged to adhere to the treatment when any of the indicators in step 16 or 5a indicated one would expect increased Events.

FIGS. 6A-6D outline other related embodiments, in equation form, where a genetic marker or gene expression level is determined to predispose test subjects to IBD or Events in test subjects, after going through the steps 1 to 12 above; one can go through steps 13 to 22 above for an individual Y with the marker or gene expression level, and determine if increased frequency of Events is dependent on qTht (6A-6B) or TrtmtA (6C-6D). The same treatment in steps 21 and 22can be used, if so. If not, one can modify TrtmtA for an individual and/or substitute a drug, supplement or another therapy for qTht for an individual (individual Y), and if determined to be effective in ameliorating IBD or reducing frequency of Events, perform similar monitoring of individual Y's adherence to the treatment regimen, by monitoring the same indicators as in steps 13to 20 and/or 5a and 6a. Again, where these indicators indicate individual Y is not adhering to the treatment regime, messages can be sent instructing adherence, and the importance of doing so. Again, the various indicators (like VOCsLVL1−x; B-VOCsLvL1−x; AvBrM; AvBrH) can also serve as a verification of individual Y's adherence to the treatment regime, and whether individual Y is truthfully reporting compliance with the treatment regime. Messages can be sent about the importance of compliance with the treatment regime when the indicators predict an increase in frequency of Events; whether or not such increase is reported by individual Y.

In another related embodiment, it can be determined for an individual (individual Z) if any gene expression levels correlate with a particular diet, or with consumption of any drug, supplement or other therapy, and with increased or reduced frequency of Events. In such case, the gene expression level can be used to monitor compliance by individual Z with the diet or the other treatment regime, as described above.

Another related embodiment is to standardize messaging to test subjects and individuals. The first step in message selection, for querying the subject's condition and for instructing treatment, is establishing, initially, a testing a set of messages for each domain, and verifying that the messages are not confusing or ambiguous or difficult to understand and correctly answer. This is accomplished by determining Cronbach's Alphas for a set of messages sent to users. For a quantity which is a sum of K, components (also called testlets or items) X=Y 1+Y 2+Y 3 . . . YK, Cronbach's alpha is defined as: α= (kk−1) (1−Σi=1kσyi2σx2)

where σ2 X is the variance of the observed total scores from subjects/individuals and where σ2 Yi is the variant of component i for the responding subjects/individuals.

To apply Cronbach's alpha in formulating a database of clear questions, for each test subject and user, one compares the sum of items' variance (through the whole set of responses from test subjects and users) to the variance of the sum of the total test scores.

If the sum of items' variance is significantly greater than the variance of the sum of the total test scores, it means that the portion of the errors resulting from misinterpretation, confusion, misunderstanding or related reasons is large, and the status the questions are designed to determine is unreliable. In such cases, the questions need to be reformulated and the new questions need to be tested for reliability using Cronbach's alpha again.

The invention has been described broadly and generically herein. Each of the narrower species and subgeneric groupings falling within the generic disclosure also form part of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A computer-implemented method of managing gastrointestinal health of a user, the method comprising:
receiving, with a local computing device of the user, a user health profile;
receiving, with one or more of the local computing device or a remote server in communication with the local computing device, generally applicable health information;
conveying to the user, via the local computing device, a treatment plan for the user based on the user health profile and the generally applicable health information;
logging, with one or more of the local computing device or the remote server, one or more of the user's food intake, drug intake, a symptom occurrence, or self-reported treatment plan compliance;
verifying, with the local computing device or the remote server, one or more of an effectiveness of or a user compliance with the treatment plan based on the logged one or more of the user's food intake, drug intake, symptom occurrence, or self-reported treatment plan compliance; and
automatically messaging the user, with the local computing device, to reinforce user compliance with the treatment plan.

2. The computer-implemented method of claim 1, further comprising generating, with one or more of the local computing device or the remote server, the treatment plan for the user based on the user health profile and the generally applicable health information.

3. The computer-implemented method of claim 2, wherein generating the treatment plan for the user comprises mining the generally applicable health information, with one or more of the local computing device or the remote server, to identify at least one treatment, technique, health tip, or health insight applicable to the user based on the user health profile.

4. The computer-implemented method of claim 3, wherein conveying to the user the treatment plan comprises conveying to the user, via the local computing device, the identified at least one treatment, technique, health tip, or health insight applicable to the user.

5. The computer-implemented method of claim 3, wherein the at least one treatment, technique, health tip, or health insight applicable to the user is identified using a machine learning algorithm.

6. The computer-implemented method of claim 1, wherein receiving the user profile comprises receiving user responses to a health questionnaire provided to the user by the local computing device.

7. The computer-implemented method of claim 1, wherein the user health profile comprises baseline information for one or more of user food consumption, drug consumption, symptom frequency, daily activities, health objectives, or a gastrointestinal health profile.

8. The computer-implemented method of claim 1, wherein the user health profile comprises a user gastrointestinal health profile comprising past and present user digestive ailments.

9. The computer-implemented method of claim 8, wherein the past and present digestive ailments comprise user status for one or more of irritable bowel syndrome (IBS), inflammatory bowel disease (IBD), frequent diarrhea, high frequency flatulence, high frequency bowel movement, or baseline frequency of symptomatic events.

10. The computer-implemented method of claim 1, wherein the user health profile comprises a user gastrointestinal health profile comprising a user microbiome profile.

11. The computer-implemented method of claim 10, further comprising generating, with one or more of the local computing device or the remote server, the user microbiome profile based on at least one fecal sample of the user.

12. The computer-implemented method of claim 1, wherein the user health profile comprises one or more of a user breath gas profile or a user fecal gas profile.

13. The computer-implemented method of claim 1, wherein the generally applicable health information comprises one or more of health-related information or unstructured medical narratives.

14. The computer-implemented method of claim 1, wherein the generally applicable health information is provided in a database suitable for data-mining.

15. The computer-implemented method of claim 1, wherein logging the one or more of food intake, symptoms, or user reported treatment plan compliance of the user further comprises logging updates to one or more of a user microbiome profile, breath gas profile, or fecal gas profile.

16. The computer-implemented method of claim 1, further updating the treatment plan in response to one or more of the effectiveness of or the user compliance with the treatment plan.

17. The computer-implemented method of claim 16, wherein updating the treatment plan comprises identifying, with one or more of the local computing device or the remote server, a treatment of the treatment plan that is effective to reduce an occurrence of at least one symptom of the user.

18. The computer-implemented method of claim 17, wherein the treatment identified as effective to reduce the occurrence of the at least one symptom of the user comprises an increased or decreased intake of a food or a drug.

19. The computer-implemented method of claim 1, wherein the local computing device of the user comprises one or more of a smart phone or personal computer.

20. A system for managing gastrointestinal health of a user, said system comprising the local computing device of the user and the remote server in communication with the local computing device, wherein the system is provided with instructions to implement the method of claim 1.
